(19) United States
(12) Patent Application Publication
(10) Pub. No.: US 2004/0005718 A1
(43) Pub. Date: Jan. 8, 2004

(54) Method of fixing biopolymers to a substrate using magnetic beads and biopolymer measuring equipment using the method

(75) Inventor: Kazuhisa Fukushima, Musashino-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: YOKOGAWA ELECTRIC CORPORATION, Tokyo (JP)

(21) Appl. No.: 10/395,192

(22) Filed: Mar. 25, 2003

(30) Foreign Application Priority Data
Jul. 5, 2002 (JP) 2002-196723

(51) Int. Cl. C12Q 1/68

(52) U.S. Cl. 436/174; 436/526; 435/6

(57) ABSTRACT

According to the present invention, biopolymers are fixed to a substrate by attaching probe DNA to magnetic beads and fixing the above magnetic beads to the substrate by attracting them using a magnetic attraction.

As described above, since magnetic beads with probe DNA attached are fixed to the substrate by attracting them using a magnetic force, the above-mentioned magnetic beads can be fixed with a force stronger than the conventional bonding of probe DNA with the substrate. Accordingly, the magnetic beads are not in danger of exfoliation from the substrate or washing-away of probe DNA during washing treatment.

### BACKGROUND OF THE INVENTION

1. Field of the Invention

The present invention relates to a method of fixing DNA probes to a DNA substrate using magnetic beads.

2. Description of the Prior Art

In recent years, DNA hybridization equipment including DNA chips has become widely used as a revolutionary tool for comprehensively analyzing the expression of genes. The DNA chip fixes several thousand kinds of different single-chain DNA fragments (probe DNA) to a substrate as mentioned, for example, in Japanese Laying Open of Patent Application No. 2001-17166 The DNA chip is able to detect a target DNA with high sensitivity by hybridizing the fixed DNA fragments and labeled target DNA fragments.

FIG. 1 is a conceptual configuration drawing of a conventional DNA chip. The DNA chip is provided with a DNA probe spotting area 2 on substrate 1 having the area of, for example, 1 inch multiplied by 3 inches (whose material may be, for example, glass, metal, silicon or plastic). Typically, several thousand sites are provided in this DNA probe spotting area 2.

On each site, probe DNA solution is spotted using a spotter device not shown in the drawing and thus probe DNA is spot-bonded as shown in FIG. 2.

For bonding probe DNA to substrate 1, static bonding or avidin-biotin bonding or covalent bonding with a coating material on a glass substrate is used.

However, if probe DNA is to be bonded (fixed) to a substrate, it has been difficult to successfully fix the probe DNA to the substrate in its single-chain state. In the process of washing away free DNA in the solution together with the solution after hybridization, even the hybridized probe DNA often comes off from the substrate and is washed away together with the solution due to incomplete fixation. This causes attenuation of signal intensity in subsequent fluorescence measurement.

Further, ideally the single chain to be developed is fixed perpendicular to the substrate surface plane. However, this is difficult in practice and often the chain becomes curled or parallel with the surface plane as shown in FIG. 3. This may cause only partial complementary bonding of DNA and impedes hybridization with the target DNA or introduces hybridization errors, thus causing attenuation of signal intensity or generation of noise.

Similarly, in attaching protein to a protein chip substrate, it is difficult to maintain the protein shape and protein function activity.

### SUMMARY OF THE INVENTION

The present invention is intended to solve the above problems by a method of fixing biopolymers to a substrate and biopolymer measuring equipment using the method, in which probe DNA is attached to magnetic beads which are fixed to the substrate by magnetic attraction.

### BRIEF DESCRIPTION OF THE DRAWINGS

FIG. 1 is a conceptual configuration drawing of conventional DNA chips.

FIG. 2 is a drawing indicating the state of spotting probe DNA to a substrate.

FIG. 3 is a drawing illustrating the state of attaching probe DNA to a conventional DNA chip.

FIG. 4 is a drawing indicating the construction of the essential part of an embodiment of equipment for realizing a fixing method of fixing biopolymers to a substrate concerning the present invention.

FIG. 5 is a drawing illustrating the status in which DNA is radially attached to the surface of a magnetic bead.

FIG. 6 is a drawing illustrating an array of the site on a substrate.

FIG. 7 is a drawing illustrating the arrangement of an electromagnet array.

FIG. 8 is a drawing indicating the status of target DNA movement.

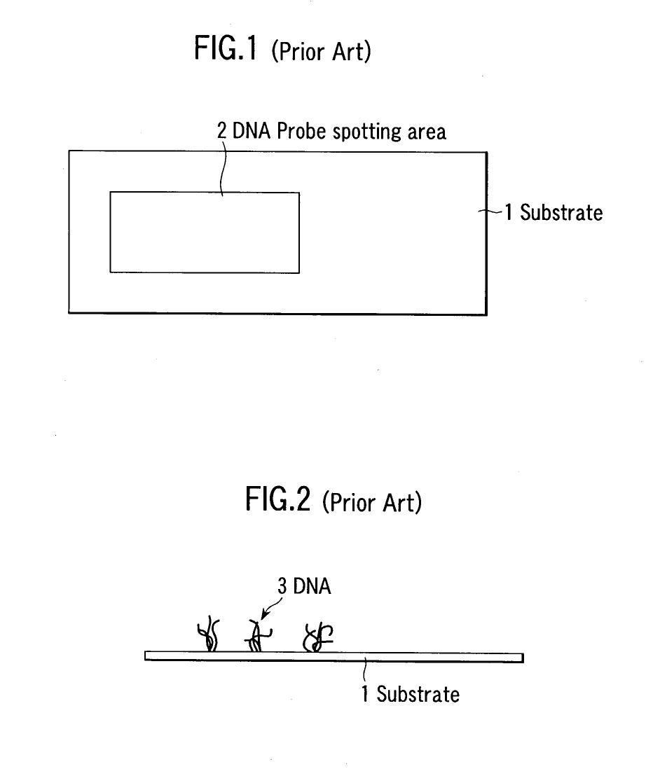

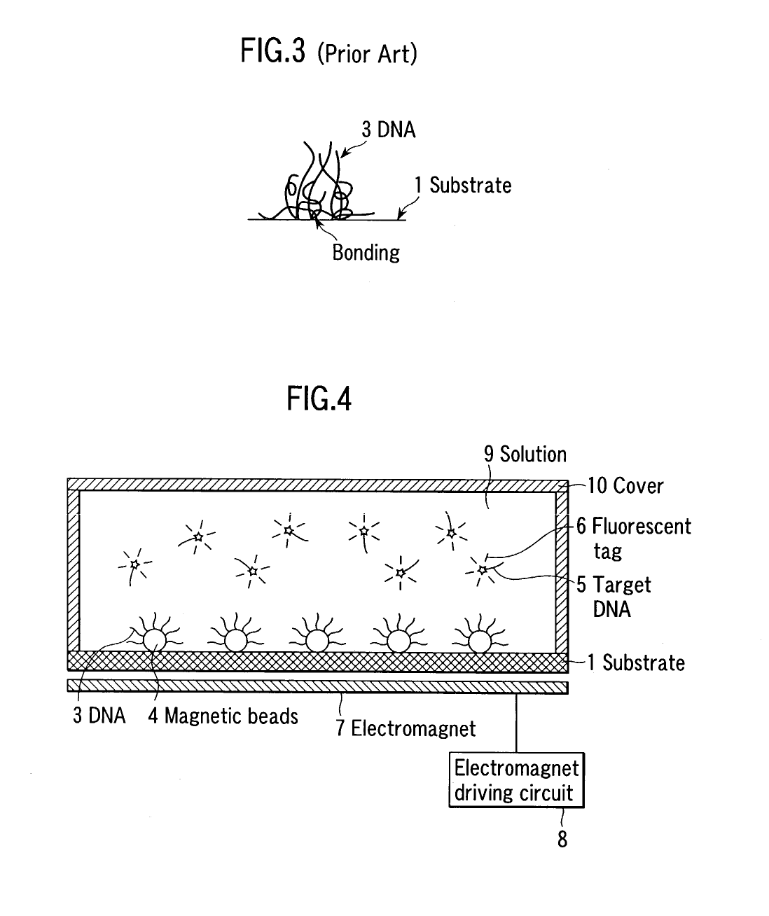

### DETAILED DESCRIPTION OF THE PREFERRED EMBODIMENTS

The present invention is described below in detail with reference to the drawings. FIG. 4 is a drawing indicating the construction of the essential part of an embodiment of equipment for realizing a method of fixing biopolymers to a substrate concerning the present invention. In this embodiment, biopolymers will be described using DNA as an example.

In FIG. 4, numeral 1 denotes the substrate of a DNA chip, numeral 4 magnetic beads to which DNA (also called probe DNA) 3 is attached, numeral 5 target DNA, numeral 6 fluorescent tags, numeral 7 an electromagnet array, numeral 8 an electromagnet driving circuit, numeral 9 a solution, and numeral 10 a cover.

Substrate 1 is formed with a nonmagnetic material, such as glass, silicon, or plastic. Cover 10 is formed with a nonmagnetic and transparent material such as glass and is mounted to substrate 1 sticking closely to it so that solution 9 is not spilt from the substrate. However, cover 10 is not necessarily fully closed but may have a construction that is partially open to air if necessary.

Magnetic beads 4 are spherical shape beads whose surfaces or the entire beads are made of a magnetic material. Known single-chain probe DNA 3 is attached (normally stuck) to the beads.

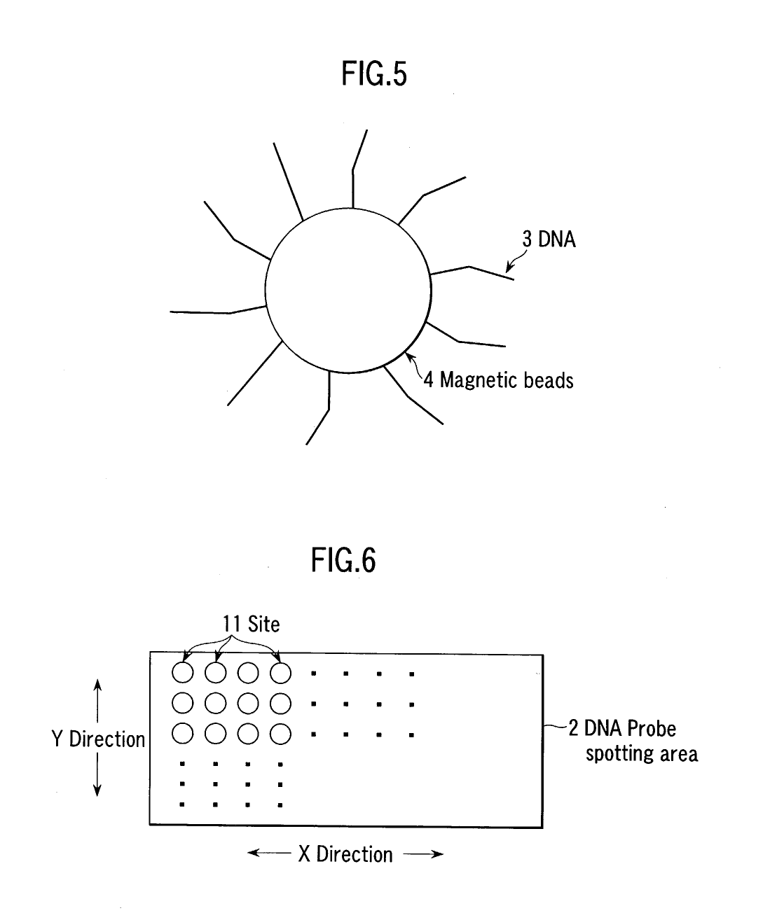

Techniques for attaching single-chain probe DNA 3 to the surface of magnetic beads 4 are well known and thus probe DNA 3 can be attached to magnetic beads strongly enough. As shown in the cross-sectional view of FIG. 5, techniques for radially attaching single-chain DNA 3 to the surface of magnetic beads 4 are also well known.

Known DNA 3 is attached to the surfaces of magnetic beads 4 for each kind of DNA 3. A plurality of sites 11 arranged as an array, for example as shown in FIG. 6, is provided on substrate 1, and magnetic beads 4, to which probe DNA 3 is attached, are placed on each site respectively.

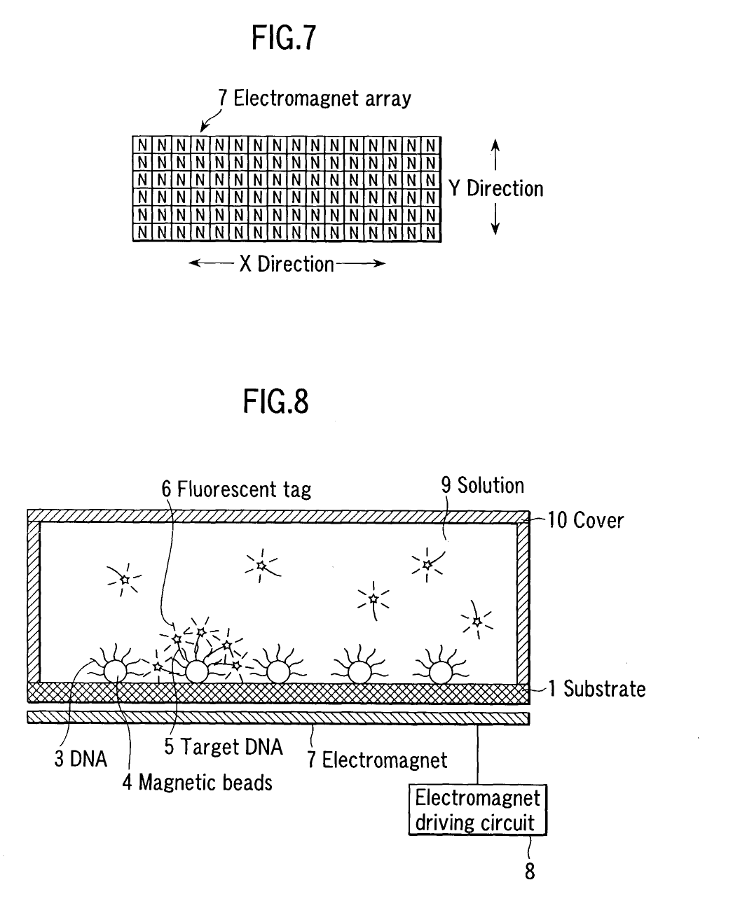

Magnetic beads 4 placed on each site are attracted towards the substrate surface by the magnetic force of an electromagnet array 7 positioned on the opposite side surface of substrate 1. Electromagnet array 7 is composed of a plurality of electromagnets that can be individually driven by electromagnet driving circuit 8 and, as shown in FIG. 7, arranged as an array corresponding to the array of sites. Each electromagnet is arranged so that its N pole (or S pole) faces the surface of substrate l and is positioned directly under each site 11.

Fluorescent tags 6 are stuck to each target DNA 5. In the initial state, each target DNA 5 is floating in solution 9 as shown in FIG. 4. Fluorescent tags 6 are utilized in target DNA 5 detection carried out after hybridization.

Operations concerning the equipment of such a configuration will now be described. First, magnetic beads 4 or magnetic material coated magnetic beads 4, on each of whose surfaces a type of known single-chain DNA 3 is attached, are prepared.

For each type of DNA 3, different magnetic beads 4 are placed on each site 11 of substrate 1 respectively. Next, at each site, magnetic beads 4 are attracted towards the electromagnet using a magnetic force by driving electromagnet 7. This enables magnetic beads 4 to be fixed to substrate 1 more strongly than by conventional sticking methods.

Next, cover 10 is mounted on substrate 1 and solution 9 containing target DNA 5 (also single-chain) is poured into the inside of the cover. Target DNA 5 with fluorescent tag floating in the solution moves about due to its own Brownian movement and if it happens to enter the vicinity of a complementary bonding mate (probe DNA 3), it hybridizes with the probe DNA 3. FIG. 8 is a drawing illustrating probe DNA 3 hybridized with target DNA 5. After hybridization, solution 9 and target DNA 5 that did not perform complementary bonding with probe DNA 3 are washed away.

In this case, since magnetic beads are strongly attracted towards the surface of substrate 1 by a magnetic force, there is no possibility of magnetic beads 4 exfoliating from substrate 1 due to washing as happened in the past.

Target DNA 5 bonded with probe DNA 3 can be detected with fluorescence reading equipment not shown in the drawing.

The present invention is not limited hereupon to the above-mentioned embodiment, but may include many further changes and versions without departing from the scope of spirit thereof.

For instance, although magnetic beads 4 to which DNA 3 are attached in the above embodiment are described as an example, the present invention can also be applied to such a bead to which biopolymers including oligo-DNA fragments or protein are attached, not limited to only DNA.

Further, although electromagnet array 7 is used as the magnetic attraction generating means in the above embodiment, a magnet array as shown in FIG. 4 can also be configured using permanent magnets.

As described above, there are the following effects according to the present invention:

Since magnetic beads to which probe DNA is attached are fixed to the substrate by being attracted by a magnetic force, the magnetic beads can be fixed to the substrate by a stronger force than the conventional bonding of probe DNA with the substrate. Accordingly, the magnetic beads do not exfoliate from the substrate and probe DNA is not washed away during washing treatment.

What is claimed is:

1. A method for fixing biopolymers to a substrate using magnetic beads, in which the magnetic beads with biopolymer fragments attached are arranged on the surface of a substrate and then said magnetic beads are attracted and fixed to the surface of said substrate by making a magnetic attraction act upon said magnetic beads from the opposite side surface of said substrate.

2. A method for fixing biopolymers to a substrate using magnetic beads in accordance with claim 1, wherein DNA or an oligo-DNA fragment or protein is used as said biopolymers.

3. A method for fixing biopolymers to a substrate using magnetic beads in accordance with claim 1 or claim 2, wherein each type of said biopolymers is attached to said each magnetic bead and said each type of magnetic bead is arranged in an array on said substrate.

4. Biopolymer measuring equipment for detecting the presence of a specific target biopolymer fragment using hybridization; comprising
a vessel, composed of a substrate and a cover mounted closely sticking to the substrate, in which magnetic beads with biopolymer fragments attached are arranged on said substrate and a solution containing target biopolymers is poured, and
a magnetic attraction generating means positioned in the opposite side of said substrate;
and being configured so that said magnetic beads are fixed to the surface of said substrate by being attracted by the magnetic attraction of said magnetic attraction generating means.

5. Biopolymer measuring equipment in accordance with claim 4, wherein DNA or oligo-DNA fragment or protein is used as said biopolymers.

6. Biopolymer measuring equipment in accordance with claim 4 or claim 5, wherein each type of said biopolymers is attached to the surface of each of said magnetic beads and said each of magnetic beads with each type of said biopolymers attached is arranged in an array on said substrate.

7. Biopolymer measuring equipment in accordance with any of claims 4 to 6, wherein said magnetic attraction generating means is configured so that magnetic attractions individually attracting a plurality of magnetic beads are generated using permanent magnets or electromagnets arranged in an array.
